(19) United States
(12) Patent Application Publication
(10) Pub. No.: US 2026/0043091 A1
(43) Pub. Date: Feb. 12, 2026

(54) METHODS AND COMPOSITIONS FOR DETECTING THE FUNGAL PATHOGEN CERATOCYSTIS FIMBRIATA

(71) Applicant: North Carolina State University, Raleigh, NC (US)

(72) Inventors: Lina QUESADA, Raleigh, NC (US); Madison STAHR, Raleigh, NC (US)

(21) Appl. No.: 19/101,369

(22) PCT Filed: Aug. 8, 2023
(86) PCT No.: PCT/US2023/071855

§ 371 Date: Feb. 5, 2025

Related U.S. Application Data
(60) Provisional application No. 63/396,017, filed on Aug. 8, 2022.

(51) Int. Cl. C12Q 1/6895; C12Q 1/6844

(52) U.S. Cl. CPC C12Q 1/6895; C12Q 1/6844; C12Q 2600/158

(57) ABSTRACT

This invention relates to molecular diagnostic tools for C. fimbriata to monitor and manage the pathogen in sweetpotato and other crop and forest systems where C. fimbriata can cause significant disease issues. Provided herein are methods to quickly detect and identify C. fimbriata in soil, wash water, diseased plants, or other complex samples.

### CROSS-REFERENCE TO RELATED APPLICATIONS

This application claims benefit of U.S. Provisional Application No. 63/396,017, filed Aug. 8, 2022, incorporated herein by reference in its entirety.

### GOVERNMENT SUPPORT CLAUSE

This invention was made with government support under Hatch Grant No. NC02628 (Accession No. 1013382) awarded by the USDA National Institute of Food and Agriculture (USDA/NIFA) and under grant numbers TASC-2019-11 and TASC-2021-03 awarded by the USDA Foreign Agricultural Service (USDA/FAS). The government has certain rights in the invention.

### REFERENCE TO AN ELECTRONIC SEQUENCE LISTING

The content of the electronic sequence listing submitted on Aug. 8, 2023, as an XML file named “10620-104W01_ST26.xml” created on Aug. 8, 2023, and having a size of 7.87 KB, is hereby incorporated by reference in its entirety pursuant to 37 CFR 1.52(e)(5).

### BACKGROUND

Sweetpotato (Ipomoea batatas) produces more biomass and nutrients per hectare than any other food crop (Ray and Ravi 2005). The tuberous roots contain many essential vitamins and minerals such as iron, potassium, and vitamins A and C (El Sheikha and Ray 2017). These nutritional benefits have made sweetpotatoes a globally important vegetable crop with over 91 million tons of sweetpotatoes harvested worldwide (FAOSTAT 2019). This significant amount is the result of increased popularity of sweetpotato and value-added products, as well as the use of sweetpotatoes in animal feed and pet food production (Bickers 2015; Scott 1992). However, such expansion and accommodations for growing consumer demands has been challenged by the re-emergence in 2014 of black rot, a historically devastating disease of sweetpotato (Halsted 1890, Scruggs et al. 2017).

Sweetpotato black rot, caused by the soilborne fungal pathogen Ceratocystis fimbriata (Ellis and Halst.), is a field and storage disease that leads to new infections and losses occurring during all stages of the sweetpotato production cycle (Clark et al. 2009). At the beginning of production, sweetpotato slips, the vegetatively propagated vine cuttings used for field transplant, can become infected when black rot sweetpotatoes are unknowingly planted in seed beds (Harter 1913). Infested soil and crop debris in the field and seed beds can also infect sweetpotato slips, plants, and the developing harvestable storage roots, while often showing little to no above ground symptoms (Harter 1913, Parada-Rojas et al. 2021, Stahr and Quesada-Ocampo 2019). At harvest, the dry and firm black rot lesions on sweetpotato roots are typically small and can easily be missed under visual inspection when covered in soil (Halsted 1890), allowing for infected plant material to enter storage and packing facilities. In these facilities, black rot lesions can further develop and sporulate under storage conditions (Stahr and Quesada-Ocampo 2020) enabling the spread of C. fimbriata through contact, handling, and wash water used in product packing (Harter 1916, Stahr and Quesada-Ocampo 2021). Yield losses in the postharvest setting can be particularly damaging as much time, effort, and money have already been invested into the sweetpotato crop. As a result, the annual yield losses from this disease have been estimated at 60% or greater (Scruggs et al. 2017, Stahr and Quesada-Ocampo 2020).

While the reemergence of sweetpotato black rot has highlighted the need for further research into effective disease management, C. fimbriata itself has been a subject of numerous studies since the characterization of the pathogen in 1890 (Halsted 1890). Starting in the early 2000's, random amplified polymorphic DNA (RAPD), and designed minisatellite and microsatellite markers have been used to evaluate genetic diversity within populations of C. fimbriata (Santini and Capretti 2000, Barnes et al 2001, Steimel et al. 2004). Over the past two decades, multiple genetic loci, such as the Ceratocystis mating-type genes (MAT1-2), the internal transcribed spacer (ITS) region, and the mini-chromosome maintenance complex component 7 (MCM7), have also been used for species differentiation and to establish phylogenetic relationships within Ceratocystis and related genera (Baker et al. 2003, Harrington et al. 2011, de Beer et al 2014). With advancements in sequencing technologies, the first draft nuclear sequence of C. fimbriata became available in 2013 (Wilken et al. 2013) and only recently have next-generation sequencing platforms and comparative genomics led to the assembly of two separate C. fimbriata genomes (Santos et al. 2020, Fourie et al. 2020). Yet, even with the increasing number of accessible genetic tools for C. fimbriata, their focus on host differentiation and phylogenetics has resulted in a lack of molecular diagnostic tools capable of rapid and direct detection. The benefits of such a tool have already been demonstrated by the recently developed real-time polymerase chain reaction (qPCR) assays for C. lukiohia and C. huliohia, which are responsible for a recent and severe outbreak of rapid ‘Ohi’a death in Hawaii (Keith et al. 2015, Heller and Keith 2018).

What is needed in the art is a molecular diagnostic tool for C. fimbriata to monitor and manage the pathogen in sweetpotato and other crop and forest systems where C. fimbriata can cause significant disease issues. Further, what is needed is an assay that could be used to quickly detect and identify C. fimbriata in soil, wash water, diseased plants, or other complex samples.

### SUMMARY

Disclosed herein are methods of treating sweetpotatoes infected with Ceratocystis fimbriata, the method comprising amplifying nucleic acid using primers which amplify a region of SEQ ID NO: 1 and/or SEQ ID NO: 2, and detecting the amplified nucleic acid, thereby detecting Ceratocystis fimbriata, and treating the sweetpotatoes to reduce or eliminate Ceratocystis fimbriata.

Also disclosed is an isolated nucleic acid, wherein the isolated nucleic acid hybridizes with at least 5 consecutive nucleic acids, in a 5′ to 3′ or 3′ to 5′ direction, within SEQ ID NO: 1 or 2.

Further disclosed is a kit comprising at least one oligomer capable of hybridizing in a 5′ to 3′ or 3′ to 5′ direction within SEQ ID NO: 1 or SEQ ID NO: 2.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-C shows a flow diagram representing the computational and laboratory approach to identify and validate candidate diagnostic markers for Ceratocystis fimbriata. A. A reference genome was assembled and annotated. B. DNA-seq reads from secondary C. fimbriata isolate C1421, C. manginecans isolate CMW17570 and C. platani isolate CMW48038 were mapped to the reference genome, counted, normalized to counts per million, and used to filter gene candidates by presence or CPM values greater than 2 in C. fimbriata, but absence or CPM values less than 2 in the other two species. Identified gene candidates were then extracted from the genome. C. Extracted sequences were aligned to the genome using Blastn to establish gene copy number. Forward and reverse primers for each candidate gene were designed in the 200 bp flanking regions, and all diagnostic candidates were subjected to lab validation against a positive DNA panel comprised of C. fimbriata samples, and three negative panels containing other Ceratocystis species, other sweetpotato pathogens, and agricultural crops and weeds grown in North Carolina.

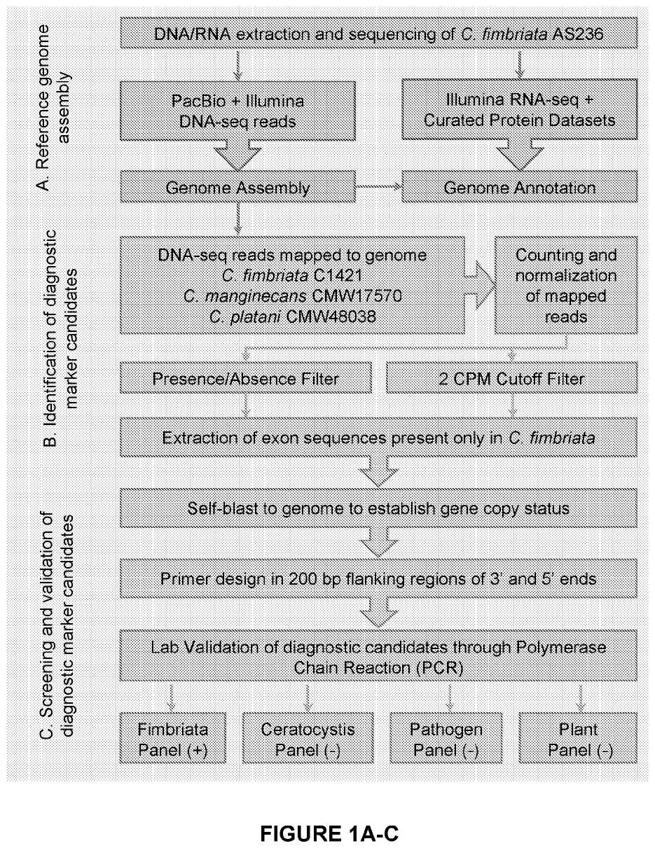

FIG. 2 shows the use of next generation sequencing data to identify species-specific regions in Ceratocystis fimbriata utilizing comparative genomics with closely related species C. manginecans and C. platani. The C. fimbriata genome panel shows two predicted genes in C. fimbriata that have less, or no mapped reads based on DNA-seq of C. fimbriata, C. manginecans and C. platani as visualized when mapped to the C. fimbriata genome. Images were generated using Geneious R10. Read color is based on Geneious default setting “Paired Distance”, and indicates read distance (yellow, green, light blue), direction (red if wrong), or if reads were unpaired (purple, dark blue). Based on gene size the following images were taken at the same zoom level for each species: T3G9—7%, T5G26—18%.

FIG. 3 shows a histogram of gene copy numbers for all Ceratocystis fimbriata diagnostic marker candidates predicted after Bowtie2 read mapping. Copy numbers was equated to the number of blast hits for a given candidate during a self-BLASTP alignment of the C. fimbriata Maker Standard gene set. Values for candidates T4G1 (483 copies) and T4G12 (597 copies) are not shown.

FIG. 4 shows gel electrophoresis images of the polymerase chain reaction amplicons of two Ceratocystis fimbriata diagnostic molecular marker genes (T3G9, T5G26) for twelve C. fimbriata samples, six other Ceratocystis species, four sweetpotato pathogens, and two plant hosts.

### DETAILED DESCRIPTION

### Definitions

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. As used herein, “complementary” or “complementarity” refers to the ability of a nucleotide in a polynucleotide molecule to form a base pair with another nucleotide in a second polynucleotide molecule. For example, the sequence 5′-A-C-T-3′ is complementary to the sequence 3′-T-G-A-5′. Complementarity may be partial, in which only some of the nucleotides match according to base pairing, or complete, where all the nucleotides match according to base pairing. For purposes of the present invention “substantially complementary” refers to 90% or greater identity over the length of the target base pair region. The complementarity can also be 50, 60, 70, 75, 80, 85, 90, 91, 92, 93, 94, 95, 96, 97, 98, 99, or 100% complementary, or any amount below or in between these amounts.

As used herein, “nucleic acid sequence” refers to the order or sequence of nucleotides along a strand of nucleic acids. In some cases, the order of these nucleotides may determine the order of the amino acids along a corresponding polypeptide chain. The nucleic acid sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc. It may include modified bases, including locked nucleic acids, peptide nucleic acids and others known to those skilled in the art.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “primer” is a nucleic acid that contains a sequence complementary to a region of a template nucleic acid strand and that primes the synthesis of a strand complementary to the template (or a portion thereof). Primers are typically 18-20 base long, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified.

By “capture sequence,” which is also referred to herein as a “second nucleic acid sequence” is meant a sequence which hybridizes to the target nucleic acid and allows the first nucleic acid sequence, or primer sequence, to be in close proximity to the target region of the target nucleic acid.

A “target region” is a region of a target nucleic acid that is to be amplified, detected or both.

The “Tm” (melting temperature) of a nucleic acid duplex under specified conditions is the temperature at which half of the nucleic acid sequences are disassociated and half are associated. As used herein. “isolated Tm” refers to the individual melting temperature of either the first or second nucleic acid sequence in the cooperative nucleic acid when not in the cooperative pair. “Effective Tm” refers to the resulting melting temperature of either the first or second nucleic acid when linked together.

As used herein, “amplify, amplifying, amplifies, amplified, amplification” refers to the creation of one or more identical or complementary copies of the target DNA. The copies may be single stranded or double stranded. Amplification can be part of a number of processes such as extension of a primer, reverse transcription, polymerase chain reaction, nucleic acid sequencing, rolling circle amplification and the like.

As used herein, “purified” refers to a polynucleotide, for example a target nucleic acid sequence, that has been separated from cellular debris, for example, high molecular weight DNA, RNA and protein. This would include an isolated RNA sample that would be separated from cellular debris, including DNA. It can also mean non-native, or non-naturally occurring nucleic acid. As used herein, “protein,” “peptide,” and “polypeptide” are used interchangeably to denote an amino acid polymer or a set of two or more interacting or bound amino acid polymers.

As used herein, “stringency” refers to the conditions, i.e., temperature, ionic strength, solvents, and the like, under which hybridization between polynucleotides occurs. Hybridization being the process that occurs between the primer and template DNA during the annealing step of the amplification process.

As used herein, “multiplex” refers to the use of PCR to amplify several different DNA targets (genes) simultaneously in a single assay or reaction. Multiplexing can amplify nucleic acid samples, such as genomic DNA, cDNA, RNA, etc., using multiple primers and any necessary reagents or components in a thermal cycler.

As used herein, a “sample” is from any source, including, but not limited to, a gas sample, a fluid sample, a solid sample, or any mixture thereof. In a preferred embodiment, the sample can be from fish, and can include, but is not limited to, scales, tissue, such as muscle or other flesh, or organs.

The term “sensitivity” refers to a measure of the proportion of actual positives which are correctly identified as such.

The term “confidence level” refers to the likelihood, expressed as a percentage, that the results of a test are real and repeatable, and not random. Confidence levels are used to indicate the reliability of an estimate and can be calculated by a variety of methods.

In certain embodiments, sequences of the present invention, including primer sequences, target sequences and internal amplification control (IAC) sequences may be identical to the sequences provided here in or comprise less than 100% sequence identity to the sequences provided herein. For instance, primer sequences, target sequences or IAC sequences of the present invention may comprise 90-100% identity to the sequences provided herein.

The terms “identical” or “percent identity,” in the context of two or more nucleic acids or sequences, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site found at ncbi.nlm.nih.gov/BLAST/or the like). Such sequences are then referred to as “substantially identical.” This definition also refers to, or applies to, the compliment of a particular sequence. The definition may also include sequences that have deletions, additions, and/or substitutions. To compensate for gene sequence diversity and to target multiple gene variants of the same genes, degenerated primer pairs (1-2 bases or approximately 5-10% alterations) are allowed.

As used herein, the term “nucleic acid” refers to a single or double-stranded polymer of deoxyribonucleotide bases or ribonucleotide bases read from the 5′ to the 3′ end, which may include genomic DNA, target sequences, primer sequences, or the like. In accordance with the invention, a “nucleic acid” may refer to any DNA or nucleic acid to be used in an assay as described herein, which may be isolated or extracted from a biological sample. The term “nucleotide sequence” or “nucleic acid sequence” refers to both the sense and antisense strands of a nucleic acid as either individual single strands or in the duplex. The terms “nucleic acid segment,” “nucleotide sequence segment,” or more generally, “segment,” will be understood by those in the art as a functional term that includes genomic sequences, target sequences, operon sequences, and smaller engineered nucleotide sequences that express or may be adapted to express, proteins, polypeptides or peptides.

The term “gene” refers to components that comprise bacterial DNA or RNA, cDNA, artificial bacterial DNA polynucleotide, or other DNA that encodes a bacterial peptide, bacterial polypeptide, bacterial protein, or bacterial RNA transcript molecule, introns and/or exons where appropriate, and the genetic elements that may flank the coding sequence that are involved in the regulation of expression, such as, promoter regions, 5′ leader regions, 3′ untranslated region that may exist as native genes or transgenes in a bacterial genome. The gene or a fragment thereof can be subjected to polynucleotide sequencing methods that determines the order of the nucleotides that comprise the gene. Polynucleotides as described herein may be complementary to all or a portion of a bacterial gene sequence, including a promoter, coding sequence, 5′ untranslated region, and 3′ untranslated region. Nucleotides may be referred to by their commonly accepted single-letter codes.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps. Similarly, any cell that “comprises,” “has” or “includes” one or more traits is not limited to possessing only those one or more traits and covers other unlisted traits.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular electrode is disclosed and discussed and a number of modifications that can be made to the electrode are discussed, specifically contemplated is each and every combination and permutation of the electrode and the modifications that are possible unless specifically indicated to the contrary. Thus, if a class of electrodes A, B, and C are disclosed as well as a class of electrodes D, E, and F and an example of a combination electrode, or, for example, a combination electrode comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures that can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to the arrangement of steps or operational flow; plain meaning derived from grammatical organization or punctuation; and the number or type of embodiments described in the specification.

### General Description

Methods of Detecting and Treating C. fimbriata

Disclosed herein are methods of detecting Ceratocystis fimbriata in various plants. Ceratocystis fimbriata is a fungus and a plant pathogen, attacking such diverse plants as the sweetpotato (black rot) and the tapping panels of the Para rubber tree (moldy rot). It has also been associated with disease in pomegranate, morning glory and carrot (Stahr et al. 2019, herein incorporated by reference in its entirety for its teaching concerning Black Rot). Because C. fimbriata is very similar to other Ceratocystis species, it is important to be able to distinguish it carefully from other species, as well as other fungi or plant pathogens such as C. betulina, C. tiliae, C. harringtonii, C. variospora, C. caryae, C. smalleyi, C. destructans, C. cacaofunesta, and C. paradoxa, for example.

It was found that two specific genes, T5G26 (SEQ ID NO: 1) and T3G9 (SEQ ID NO: 2), are very specific to C. fimbriata, so that amplifying nucleic acid within the region of SEQ ID NO: 1 and/or SEQ ID NO: 2 allows one to discriminate between C. fimbriata and other species. This amplification can be done with primers specific for SEQ ID NO: 1 and/or SEQ ID NO: 2, so that if an amplified product is detected, one of skill in the art knows that C. fimbriata is present in the sample.

In various embodiments, sample preparation (i.e., preparation of the target DNA) involves rupturing the cells (e.g., cells of the tissue or fungal spores in patient body fluid or tissue) and isolating the fungal DNA from the lysate. The DNA can also be amplified directly once released from ruptured cells or tissue. Techniques for rupturing cells and for isolation of DNA are well-known in the art. For example, cells may be ruptured by using a detergent or a solvent, such as phenol-chloroform. DNA may be separated from the lysate by physical methods including, but not limited to, centrifugation, pressure techniques, or by using a substance with affinity for DNA, such as, for example, silica beads. After sufficient washing, the isolated DNA may be suspended in either water or a buffer. In other embodiments, commercial kits are available, such as Quiagen™, Nuclisensm™, and Wizard™

(Promega), and Promegam™. Methods for isolating DNA are described in Sambrook et al., “Molecular Cloning: A Laboratory Manual”, 3rd Edition, Cold Spring Harbor Laboratory Press, (2001), incorporated herein by reference.

In various embodiments described herein, the primers and probes used for amplification of the target DNA and for detection and identification of C. fimbriata are oligonucleotides from about ten to about one hundred, more typically from about ten to about thirty or about six to about twenty-five base pairs long, but any suitable sequence length can be used. In illustrative embodiments, the primers and probes may be double-stranded or single-stranded, but the primers and probes are typically single-stranded. The primers and probes described herein are capable of specific hybridization, under appropriate hybridization conditions (e.g., appropriate buffer, ionic strength, temperature, formamide, and MgCl2 concentrations), to a region of the target DNA. The primers and probes described herein are designed based on having a melting temperature within a certain range, and substantial complementarity to the target DNA. Methods for the design of primers and probes are described in Sambrook et al., “Molecular Cloning: A Laboratory Manual”, 3rd Edition, Cold Spring Harbor Laboratory Press, (2001), incorporated herein by reference. The primers and probes described herein for use in PCR can be modified by substitution, deletion, truncation, and/or can be fused with other nucleic acid molecules wherein the resulting primers and probes hybridize specifically to the intended targets and are useful in the methods described herein for amplification of the target DNAs. Derivatives can also be made such as phosphorothioate, phosphotriester, phosphoramidate, and methylphosphonate derivatives, that specifically bind to single-stranded DNA or RNA (Goodchild, et al., Proc. Natl. Acad. Sci. 83:4143-4146 (1986)).

Also within the scope of the invention are nucleic acids complementary to the probes and primers described herein, and those that hybridize to the nucleic acids described herein or those that hybridize to their complements under highly stringent conditions. In accordance with the invention “highly stringent conditions” means hybridization at 65° C. in 5×SSPE and 50% formamide, and washing at 65° C. in 0.5×SSPE. In some illustrative aspects, hybridization occurs along the full-length of the nucleic acid.

Also included are nucleic acid molecules having about 60%, about 70%, about 75%, about 80%, about 85%, about 90%, about 91%, 92%, 93%, 94%, 95%, 96%, 97%, 98%, or 99% homology to the probes and primers described herein. Determination of percent identity or similarity between sequences can be done, for example, by using the GAP program (Genetics Computer Group, software), and alignments can be done using, for example, the ClustalW algorithm (VNTI software, InforMax Inc.). A sequence database can be searched using the nucleic acid sequence of interest. Algorithms for database searching are typically based on the BLAST software (Altschul et al., 1990). In some embodiments, the percent identity can be determined along the full-length of the nucleic acid.

Techniques for synthesizing the probes and primers described herein are well-known in the art. Primers and probes can also be made commercially (e.g., CytoMol, Sunnyvale, CA or Integrated DNA Technologies, Skokie, IL). Techniques for purifying or isolating the probes and primers described herein are well-known in the art. The primers and probes described herein can be analyzed by techniques known in the art, such as restriction enzyme analysis or sequencing, to determine if the sequence of the primers and probes is correct. In various embodiments of the methods and compositions described herein, the probes and primers can be labeled, such as with fluorescent compounds, radioactive isotopes, antigens, biotin-avidin, colorimetric compounds, or other labeling agents known to those of skill in the art, to allow detection and quantification of amplified DNA, such as by Real-Time PCR. In illustrative embodiments, the labels may include 6-carboxyfluorescein (F AM™), TET™ (tetrachloro-6-carboxyfluorescein), JOE™ (2,7-dimethoxy-4,5-dichloro-6-carboxyfluorescein), VIC™, HEX (hexachloro-ó-carboxyfluorescein), TAMRA™ (6-carboxy-N,N,N′,N′-tetramethylrhodamine), BHQ™, SYBR® Green, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein, Oregon Green 488, Oregon Green 500, Oregon Green 514, Pacific Blue, REG. Rhodamine Green, Rhodamine Red, ROX, and/or Texas Red.

Specificity of the probes and primers described herein was demonstrated by testing hybridization of the probe and primers sets against different species of Ceratocystis (Table 2 and Table 3). There were no cross-over reactions or cross-over detection noted for any of the tested probe and primer sequences. Thus, the methods and compositions {e.g., primers and probes) for amplification of C. fimbriata are highly specific and avoid co-amplification of or do not co-amplify non-specific nucleic acids.

The oligonucleotides (such as primers and probes) disclosed herein can each be from about 5 to about 100 nucleotides, from about 10 to about 50 nucleotides, from about 15 to about 35 nucleotides. or from about 20 to about 30 nucleotides. In some embodiments, the probes are at least 5-mers, 6-mers, 7-mers, 8-mers, 9-mers, 10-mers, 11-mers, 12-mers, 13-mers, 14-mers, 15-mers, 16-mers, 17-mers, 18-mers, 19-mers, 20-mers, 21-mers, 22-mers, 23-mers, 24-mers, 25-mers, 26-mers, 27-mers, 28-mers, 29-mers, 30-mers, 31-mers, 32-mers, 33-mers, 34-mers, 35-mers, 36-mers, 37-mers, 38-mers, 39-mers, 40-mers, 41-mers, 42-mers, 43-mers, 44-mers, 45-mers, 46-mers, 47-mers, 48-mers, 49-mers, 50-mers, 51-mers 52-mers, 53-mers, 54-mers, 55-mers, 56-mers, 57-mers, 58-mers, 59-mers, 60-mers, 61-mers, 62-mers, 63-mers, 64-mers, 65-mers, 66-mers, 67-mers, 68-mers, 69-mers, 70-mers, 71-mers, 72-mers, 73-mers, 74-mers, 75-mers, 76-mers, 77-mers, 78-mers, 79-mers, 80-mers, 81-mers, 82-mers, 83-mers, 84-mers, 85-mers, 86-mers, 87-mers, 88-mers, 89-mers, 90-mers, 91-mers, 92-mers, 93-mers, 94-mers, 95-mers, 96-mers, 97-mers, 98-mers, 99-mers, 100-mers or combinations thereof.

The amplification reaction described above needs reagents in order for amplification to occur. One of skill in the art can readily determine which reagents should be present in order to amplify a sample. Such reagents include, but are not limited to, PCR “Mastermix”; Taq polymerase; RNase H2 enzyme, RNase H2 enzyme buffer, and primers or labeled primers. Methods such as polymerase chain reaction (PCR, rhPCR, and RT-PCR) and ligase chain reaction (LCR) or isothermal PCR reaction may be used to amplify nucleic acid sequences directly from genomic material. For example, the PCR assay may include a number of reagents and components, including a master mix and nucleic acid dye or intercalating agent. In some embodiments, an exemplary PCR master mix may contain template genomic material, such as DNA or RNA, RNase H2 enzyme, RNase H2 enzyme buffer, PCR primers or labeled PCR primers, probes salts such as MgCl₂, a polymerase enzyme, and deoxyribonucleotides. One of skill in the art will be able to identify useful components of a master mix in accordance with the present invention.

Also disclosed are methods of treating sweetpotatoes infected with Ceratocystis fimbriata, the method comprising amplifying nucleic acid using primers which amplify a region of SEQ ID NO: 1 and/or SEQ ID NO: 2, and detecting the amplified nucleic acid, thereby detecting Ceratocystis fimbriata, and treating the sweetpotatoes to reduce or eliminate Ceratocystis fimbriata.

Methods of treating C. finbriata are known to those of skill in the art. Examples include the application of various antifungals, antimicrobials, and sanitizers. Also contemplated are natural means of controlling fungi such as changing the growing conditions (amount of water, fertilizer, shade/sun, etc.)

### Nucleic Acid Oligomers and Kits

Disclosed herein are isolated nucleic acids, wherein the isolated nucleic acid hybridizes with at least 5 consecutive nucleic acids, in a 5′ to 3′ or 3′ to 5′ direction, within SEQ ID NO: 1 or 2. This means that the isolated nucleotide hybridizes with 5 or more nucleotides within SEQ ID NO: 1, or alternatively, within SEQ ID NO: 2. The nucleotide can hybridize with 5, 6, 7, 8, 9, 10, 11, 12, 13, 14, 15, 16, 17, 18, 19, 20, 21, 22,23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides. The nucleotide can vary in length, as described above, but can be anywhere from 5 to 100 or more nucleotides in length.

In some embodiments, the isolated nucleic acid is 90% or more identical to one of the primers disclosed in Table 5. As discussed above, the nucleic acid can be 90, 91, 92, 93, 94, 95, 96, 97, 98, 99, or 100% identical to any of SEQ ID NOS: 3, 4, 5, and 6.

It is noted that SEQ ID NO: 3 and 4 are an exemplary primer pair which can amplify SEQ ID NO: 2 (gene T3G9). SEQ ID NOS: 5 and 6 are an exemplary primer pair which can amplify SEQ ID NO: 1 (gene T5G26).

Also disclosed is a kit. The kit can comprise, at a minimum, at least one oligomer capable of hybridizing in a 5′ to 3′ or 3′ to 5′ direction within SEQ ID NO: 1 or SEQ ID NO: 2. The design of these oligomers are discussed in detail above. In one example, this sequence can be SEQ ID NO: 3, 4, 5, or 6. The kit can comprise more than one oligomer, so that it includes SEQ ID NOS: 3 and 4, for example, or SEQ ID NOS: 5 and 6. In another embodiment, it can include all of SEQ ID NOS: 3, 4, 5, and 6. The oligomers can be primers or probes. The kit can optionally include at least two primers and a probe.

The kit can further include additional reagents needed for amplification or detection, such as one or more of labeling reagents, buffers, or enzymes. For example, the kit can further include DNA polymerase or reverse transcriptase. These are discussed in more detail above.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Development of Species-Specific Primers Using Long- and Short-Read Technology for the Fungal Pathogen, Ceratocystis fimbriata Materials and Methods

Fungal materials, DNA/RNA extraction, and sequencing. Actively growing cultures of Ceratocystis fimbriata, C. manginecans, and C. platani were used for genetic material in this study. Isolate descriptions can be found in Table 1. To prepare for DNA or RNA extraction, isolates were first grown on carrot agar (16% carrot juice [Bolthouse Farms Inc., Bakersfield, CA], 16 g select agar per L) amended with rifampicin and ampicillin (20 mg/L), for 10 days. When cultures were mature, three 5-mm square agar plugs were excised from the leading edge of each culture and were placed in a deep petri plate (100×25 mm) containing 10% clarified carrot broth (10% Bolthouse Farms carrot juice, filtered). Broth cultures were wrapped shut with Parafilm (Bemis Company, Neenah, WI) to prevent contamination and placed in a 25° C. incubator without light or agitation for up to 14 days.

Fungal DNA or RNA was extracted from mature broth cultures. To extract nucleic acids, agar plugs were removed from each culture using sterilized tweezers. Mycelial tissue was then vacuum filtered, and 1 gram of tissue was placed into 2-mL centrifuge tubes with a combination of 2-mm ceramic and fine, acid-washed glass beads. All tubes were then lyophilized (−50° C. and 0.040 mBar; Labconco, Kansas City, MO) for 72 hours, and the tissue was immediately disrupted following lyophilization in an Omni Bead Ruptor 24 (Omni International Inc., Kennesaw, GA) for two 30s cycles with a 2s dwell period. DNA was extracted from ground tissue using phenol/chloroform methods, following an adapted protocol of Kaur et al. (2018), or RNA was extracted using a Qiagen RNeasy Mini kit (Qiagen Sciences, Germantown, MD) following manufacturer instructions. Genetic material was submitted to the Genomic Sciences Laboratory (GSL) at North Carolina State University for library preparation and sequencing. Library preparation for PacBio sequencing was completed using the SMRTbell template library prep kit (15-20 Kb fragments; Pacific Biosciences of California, Menlo Park, CA). Illumina library preparation was completed with Illumina Truseq Nano DNA library prep kit (Illumina Inc., San Diego, CA) or NEBNext Ultradirectional RNA library prep kit (New England Biosciences, Ipswich, Massachusetts). Long-read DNA and short-read (paired-end, 150 bp read length) DNA and RNA sequences were obtained for C. fimbriata isolate AS236 using PacBio Sequel and Illumina NextSeq 500, respectively. Additional short-read DNA sequences (paired-end, 150 bp read length) using the Illumina NovaSeq 6000 were obtained from C. fimbriata isolate C1421. Finally short-read DNA sequences from C. manginecans and C. platani were obtained using Illumina MiSeq (MiSeq V2 chemistry, paired-end, 250 bp read length). All raw sequencing files generated for genome assembly and annotation were deposited in the Sequence Read Archive (SRA) database under BioProject PRJNA705194.

Hybrid assembly and annotation of the Ceratocystis fimbriata genome. Raw PacBio Sequel reads from C. fimbriata isolate AS236 were assembled by Canu (v.1.8; Koren et al. 2017). Reads less than 1,000 bp were removed prior to read correction, trimming, and assembly by Canu. The assembly was performed using a maximum estimated genome size of 40 Mbp. Trimmomatic (v.0.32; Bolger et al. 2014) was used to remove adaptor sequences and filter paired end reads for quality from Illumina DNA-seq reads from AS236. The paired end Illumina DNA reads were then used to polish the Canu assemblies using three iterations of Racon (v.1.4.0; Vaser et al. 2017) and Pilon (v.1.23; Walker et al. 2014). The completeness of the genome assembly (GenBank accession JAJQWW000000000; FigShare DOI 10.6084/m9.figshare.17131646) was assessed using BUSCO (v 3.1.0; Waterhouse et al. 2017, Simao et al. 2015) through comparison to the fungi-specific lineage database.

To assemble the transcriptome of C. fimbriata, paired end Illumina RNA-seq reads from isolate AS236 were trimmed and filtered using trimmomatic. The cleaned RNA reads were entered into STAR (v.2.7.0a; Dobin and Gingeras, 2016) to generate a genome index using the reference C. fimbriata Canu genome assembly, and reads were assembled using Trinity (v.2.4.0; Grabherr et al. 2013). Iterative runs of the MAKER (v.2.31.9; Campbell et al. 2014b) annotation pipeline were then used to annotate the C. fimbriata genome. Briefly, the first iterative run of MAKER was used to perform repeat masking using RepeatMasker within MAKER, and evidence alignment based on the publicly available, highly curated fungal protein datasets from Aspergillus nidulans (Aspergillus Genome Database strain FGSC_A4; Cerqueira et al. 2014), Neurospora crassa (Ensembl assembly GCA_000182925.2; Galagan et al. 2003, Yates et al. 2020), Saccharomyces cerevisiae (Saccharomyces Genome Database reference strain S288C; Cherry et al. 2012), and Ustilago maydis (Ensembl assembly GCA_000328475.2; Ho et al. 2007, Yates et al. 2020). This initial run resulted in a GFF (general feature format) file containing the location of masked regions, transcript alignments, and protein alignments that was used in the subsequent run of MAKER. Transcript-derived gene models generated during the initial MAKER run, with annotation edit difference (AED) values greater than or equal to 0.2, were considered to be high-quality and were subsequently used to train the ab initio gene prediction programs SNAP (v.2.31.9; Korf 2004) and AUGUSTUS (v3.3.3; Stanke and Waack 2003). After training the gene prediction programs, MAKER was run for a final time using SNAP and AUGUSTUS to predict all possible gene-models, which comprised the MAKER max gene set (Campbell et al. 2014a).

Transcript and protein fasta files and a GFF file with all gene predictions and evidence data for the MAKER max gene set were generated using MAKER accessory scripts fasta merge and gff_merge. Predicted gene-models that encoded PFAM domains were then identified using hmmscan as part of the package HMMER (v.3.2.1; Eddy 2018). The hmmscan output, the MAKER max transcript and protein fasta files, and the MAKER max GFF file were then used to filter the max gene set to remove low quality gene predictions that lacked both transcript evidence and evidence that the predicted gene encoded a PFAM domain. This was done using the custom scripts generate_maker_standard_gene_list.pl, get subset_of_fastas.pl, and create_maker_standard_gff.pl from Bowman et al. (2017) (available on the Childs Lab GitHub repository). The transcripts of all removed gene-models were then evaluated for the presence of signal peptides using SignalP (v4.1; Peterson et al. 2011). Transcripts found to have signal peptide motifs were aligned to publicly available fungal protein sequences in the NCBI database through a BLASTx (v2.9.0) search. Transcripts with signal peptides that also had at least one BLAST hit with an e-value better than 0.00001 were then rescued from the removal process and added back into the gene set. These combined steps resulted in the MAKER standard gene set (Campbell et al. 2014a), which contained all gene-models that possessed transcript evidence, PFAM domain evidence, or signal peptide motifs.

Finally, the MAKER standard gene set was aligned to the highly curated and characterized, publicly available fungal protein datasets from A. nidulans (Aspergillus Genome Database strain FGSC_A4; Cerqueira et al. 2014), N. crassa (Ensembl assembly GCA_000182925.2; Galagan et al. 2003, Yates et al. 2020), S. cerevisiae (Saccharomyces Genome Database reference strain S288C; Cherry et al. 2012), and U. maydis (Ensembl assembly GCA_000328475.2; Ho et al. 2007, Yates et al. 2020) using BLAST-P (minimum e-value cutoff of 0.00001). These BLAST results were compared to a text file containing the genes from these four curated species with detailed gene descriptions. The detailed descriptions for any genes that the C. fimbriata gene-models aligned to, along with the PFAM domain annotations generated previously in the MAKER pipeline, were then used to compile a descriptive, functional annotation of all C. fimbriata gene-models (FigShare DOI 10.6084/m9.figshare.17131646).

Bioinformatics approach to identify diagnostic markers. To predict unique regions of the C. fimbriata genome that could be used for species-specific diagnostics, Illumina DNA reads from C. fimbriata isolate C1421, C. manginecans, and C. platani were trimmed and filtered for quality using Trimmomatic as done previously. Bowtie2 (v2.2.6; Langmead et al. 2018) was then used to build an index of the assembled C. fimbriata AS236 genome. Cleaned reads of the other three isolates were then mapped to the genome also using Bowtie2, to indicate which regions were present across the C. fimbriata species, as marked by the mapping of reads from secondary C. fimbriata isolate C1421, but absent in other closely related Ceratocystis species. The final GFF file generated from the MAKER annotation was converted to a GTF file using gffread in the program Cufflinks (v2.2.1; Trapnell et al. 2010). HTSeq (v0.13.5; Anders et al. 2014) was then used to count the number of reads mapped in the Bowtie2 alignment files to every exon within the C. fimbriata GTF file. The read counts from HTSeq were normalized to counts per million (CPM) using the following equation:

$${CPM}_{i} = \frac{{X}_{i}}{N} \times 1 {0}^{6}$$

Where Xᵢ is the number of mapped reads and N is the number of fragments sequenced. The exons were then separately filtered in two ways to identify exons unique to C. fimbriata. First, the exons were filtered to only keep exons present in C. fimbriata based on any CPM value greater than 0, but absent from C. manginecans and C. platani based on CPM values equal to 0. Second, the exons were filtered using a CPM cut off of 2; to identify and keep exons that had mapped read counts from C. fimbriata greater than 2 CPM but mapped read counts from C. manginecans and C. platani less than 2 CPM. A non-redundant list of exons from each filtering method was compiled to form a list of C. fimbriata diagnostic marker candidates.

Primer design and validation with PCR. A self-BLAST was conducted through alignment of the C. fimbriata protein fasta file to itself using BLAST-P. The BLAST results for all diagnostic markers on the candidate list were then extracted from the BLAST output to establish gene copy number of the diagnostic marker candidates. Next, fasta sequences of the exons in the diagnostic marker list, with 200 bp flanking regions in the 3′ and 5′ ends of each exon, were extracted from the genome using extractseq in EMBOSS (v6.6.0; Ison et al. 2011). The extracted fasta sequences were then used for primer design in Geneious (vR10; Biomatters Ltd., Auckland, New Zealand). Within Geneious, molecular markers with fasta sequences less than 500 bp in size, indicating the original exon was less than 100 bp, were removed from the candidate list. For the remaining candidates, forward and reverse primers with annealing temperatures between 52 and 57° C. were designed to fall within the 200 bp regions flanking each exon using Primer3 (2.3.7; Koressaar and Remm 2007) within Geneious. Due to size limitations of polymerase chain reaction (PCR) amplification, additional primer sets were designed to fall within the exon sequence of the molecular markers when exon sequences were greater than 1,500 bp.

The resulting primer pairs were ordered through Integrated DNA Technologies (Coralville, Iowa) and validated by PCR against a series of DNA panels that included multiple C. fimbriata isolates, other Ceratocystis species, additional sweetpotato pathogens, and 12 plant species including known hosts sweetpotato (variety Covington) and morning glory (Qpomoea nil, variety Scarlett O'hara) (Table 1, 2, 3). PCR reactions, completed according to manufacturer's guidelines, contained 1 Promega GoTaq G2 Hot Start Green Master Mix (Promega, Durham, NC), 10 μM of forward and reverse primers, and 10 μM of DNA. The PCR program used for amplification included a denaturation step at 94° C. for 3 min followed by 30 cycles of 94° C. for 30 s, 55° C. for 30s, and 72° C. for 30s and a final elongation step of 72° C. for 10 minutes. Products were visualized through gel electrophoresis on a 2.0% agarose gel containing 0.2 μg/mL ethidium bromide, run at 65 volts for 1 hour, followed by detection with a Geldoc Imager (Bio-Rad, Hercules, California) using Image Lab Software (v4.1, Bio-Rad). Product sizes were estimated by comparison with a 100-bp DNA ladder (New England Biolabs, Ipswich, Massachusetts). Diagnostic candidates that failed to amplify a consistently sized PCR amplicon in all 25 C. fimbriata isolates tested or that amplified in any of the other Ceratocystis species, sweetpotato pathogens or in any of the plant species screened were eliminated. Diagnostic markers still remaining after validation were considered highly specific to C. fimbriata.

### Results

Hybrid assembly and annotation of the Ceratocystis fimbriata genome. The assembly of PacBio long-read sequences using Canu, followed by polishing and correction with Illumina short-read sequences through Pilon and Racon resulted in a hybrid genome assembly for C. fimbriata with an approximate size of 31.6 Mb (Table 5). The assembly was divided across 18 contigs that had a contig N50 of 3.8 Mb. To assess the quality of the genome assembly, benchmarking universal single-copy orthologs or BUSCO analysis was done through comparison to the fungi-specific lineage database. During this analysis, 98.4% of the 758 core genes were identified in the genome assembly. Of these core genes, 745 complete-single copy BUSCOs, 1 complete-duplicated BUSCO, and 2 fragmented BUSCOs were found.

Following genome assembly, MAKER-P was used for genome annotation using C. fimbriata RNA sequences and curated fungal protein datasets from four well-characterized species (A. nidulans, N. crassa, S. cerevisiae, and U. maydis) as protein evidence. The MAKER annotation predicted a total of 7,840 gene models in the Maker max gene set. All predicted gene models were then screened for evidence of transcript or protein family domain support to remove any low-quality gene predictions. During this process 1,517 gene models were removed due to lack of supporting evidence. Transcripts from the removed gene models were additionally screened using Signal-P and a BLASTx alignment to publicly available fungal protein sequences in the NCBI database. This process indicated 267 of the removed genes had evidence of a signal peptide domain, of which 162 also aligned to at least one publicly available protein sequence. Genes with transcripts with signal peptide domain and BLASTx alignment support were then added back into the Maker Standard gene set, which contained 6,481 gene models supported by transcript, protein family domain, or signal peptide domain evidence. An overview of general genome assembly and annotation statistics can be found in Table 4.

Descriptive, functional annotations of all gene models within the Maker Standard gene set were compiled from the descriptive annotations of homologous genes from A. nidulans, N. crassa, S. cerevisiae, and U. maydis as well as the description of protein family domains found within the gene models (sup. file 1; FigShare DOI 10.6084/m9.figshare.17131646). Through this process, approximately 83% of the C. fimbriata predicted genes were given a descriptive annotation that described a defined or putative function. An additional 8% of gene models did not possess a known function but were found to have at least one homolog amongst the four other species compared to. Of the remaining predicted gene models, 339 were considered to be expressed genes with no known function based on transcript evidence, and 236 genes were considered hypothetical with no known function based on a lack of transcript evidence.

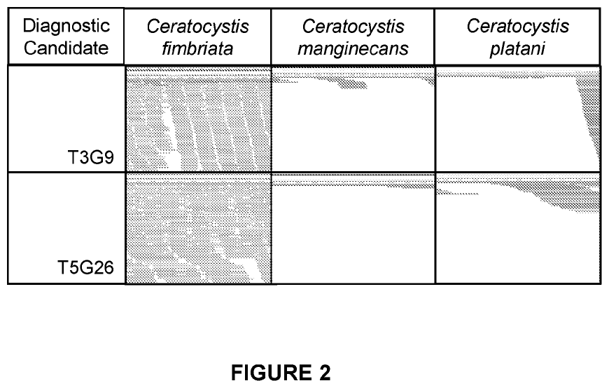

Identification and validation of diagnostic marker candidates. When Illumina DNA-seq reads were mapped to the assembled C. fimbriata reference genome, 24,138,861 reads from C. fimbriata isolate C1421 had an overall alignment rate of 95.11%, 3,933,921 reads from C. manginecans had an overall alignment rate of 91.46%, and 4,734,325 reads from C. platani had an overall alignment rate of 89.14%. After counting the number of reads mapped to each exon defined in the reference GTF file using HTseq, the read counts were converted to counts per million and used as the computational basis for identifying genes with exons present only within C. fimbriata (FIG. 2). When filtering for genes with unique exons by presence in C. fimbriata based on CPM values greater than zero, but absence in C. manginecans and C. platani based on CPM values equal to zero, 127 candidate genes with single or multiple exons unique to C. fimbriata were identified. When filtering for genes with unique exons by presence in C. fimbriata based on CPM values greater than or equal to two, but absence in C. manginecans and C. platani based on CPM values less than two, 110 candidate genes with single or multiple exons unique to C. fimbriata were identified. The non-redundant list of suitable gene candidates for diagnostic markers was compiled from both of these methods to produce a total list of diagnostic marker candidates with 148 genes.

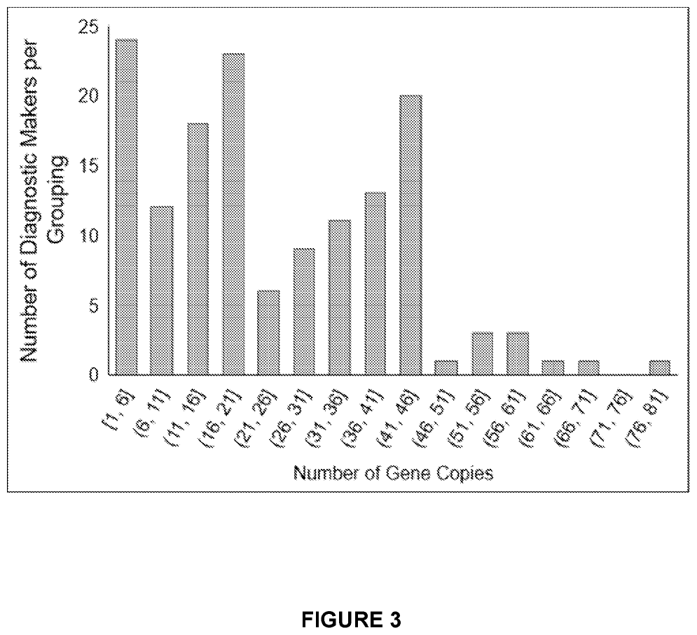

The self-BLAST-P analysis of all predicted C. fimbriata gene models enabled the identification of single or highly multi-copy genes within the marker candidate list. A total of 6 candidates were found to be putative single-copy genes, and 35 candidates were considered highly multi-copy with greater than 40 blast hits (FIG. 3). Of these multi-copy genes, 33 fell between 40 and 79 blast hits, but gene candidates T4G1 and T4G12 had 483 and 597 blast hits, respectively. The number of exons per candidate gene was also variable, and complete gene sequences across all exons were often larger than what could be amplified using PCR. Thus only the first unique exon listed in the C. fimbriata GTF reference file for each candidate gene was chosen for marker development. Once fasta sequences were extracted for the 148 candidate markers and entered into Geneious for primer development, 10 genes were removed from the marker candidate list due to the selected first unique exon sequences being less than 100 bp. Another 24 of the marker candidates had exon sequences over 1,500 bp. Thus an additional primer set was designed to fall within the exon sequence to reduce the risk of failed amplification due to size limitations of PCR products. Overall a total of 162 primer pairs for 138 marker candidates were selected for lab validation.

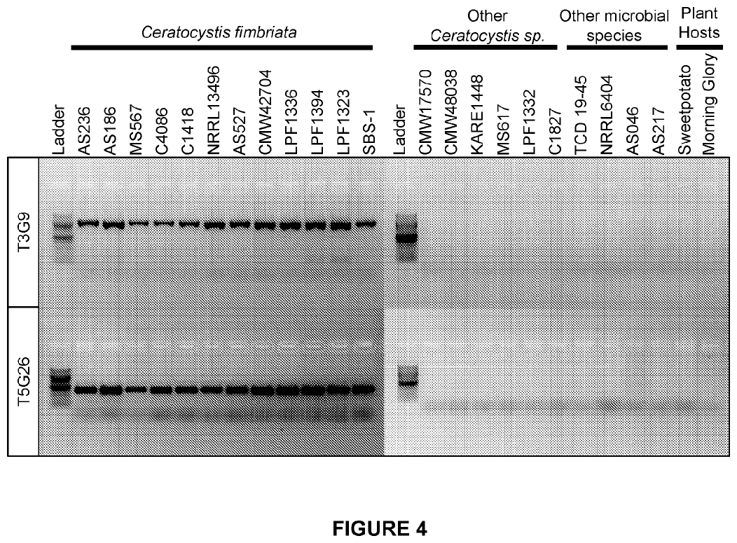

Validation of molecular markers through PCR began against a diverse panel of C. fimbriata isolates that were derived from 12 plant hosts, 15 different geographic locations, and were collected over a 20-year period (1998 to 2018; Table 1). Diagnostic markers that failed to produce consistent PCR amplicons across all 25 C. fimbriata DNA samples tested were removed from the candidate list, resulting in 33 primers pairs representing the same number of diagnostic marker candidates proceeding to further validation. These molecular markers were then tested against multiple negative panels: a panel of 11 other Ceratocystis spp. not including C. fimbriata, that were derived from 11 hosts, 10 different geographic locations, and over a 34-year period (1986 to 2020; Table 1); a panel of 13 different common sweetpotato pathogens (Table 2), which included fungi, bacteria, and nematodes; and 12 plant species including C. fimbriata plant hosts sweetpotato and morning glory (Table 3). Diagnostic markers that produced any PCR amplicons in other Ceratocystis spp. (FIG. 4), sweetpotato pathogens, or plant samples, were also removed from the candidate list, resulting in a final number of two marker candidates that were considered to be highly specific to C. fimbriata.

Of the two final candidates, diagnostic marker T3G9 had a descriptive annotation as an endonuclease that also has reverse transcriptase activity, while diagnostic marker T5G26 lacked a known function, but was considered an expressed gene based on transcript evidence (Table 6). Although none of the putative single-copy markers were within the final candidate pool, candidate T5G26 was highly multi-copy with over 40 BLASTP hits when aligned to the assembled C. fimbriata transcriptome.

### Discussion

The 31.7 Mbp C. fimbriata genome assembly produced in this study has many similarities to the two other C. fimbriata genomes currently publicly available (Fourie et al. 2020, Santos et al. 2020). The first of these genomes is based on C. fimbriata isolate CMW14799, which was derived from sweetpotato in North Carolina in 1998. Although a draft genome sequence for CMW14799 was compiled in 2013 from 454 pyrosequences (Wilken et al. 2013), an updated assembly was published in 2020 through the combined use of Illumina short-read and Nanopore long-read sequencing (Fourie et al. 2020). The second isolate of C. fimbriata to be fully sequenced and assembled was isolate LPF1912 that was derived from eucalyptus in Bahia State, Brazil, and was compiled from Illumina short-read sequences (Santos et al. 2020). These two genomes are similar in size to the present assembly, ranging from 32.08 to 31.6 Mbp, respectively, and have a nearly identical GC content of approximately 48% (Fourie et al. 2020, Santos et al. 2020). The most notable difference between the three genome assemblies is the number of contigs, which is greatly attributed to the combined use of long and short read sequencing that has become an increasingly common strategy to improve sequence assembly quality for a wide range of organisms (Elbers et al. 2018, Passera et al. 2018, Polonio et al. 2021). Without the use of long read sequencing, the assembly of LPF1912 had a total of 799 scaffolds, while the addition of Nanopore sequencing of CMW14799 led to an assembly with 16 contigs, and the use of PacBio in this study led to 18 contigs. Although this is a vast improvement in genomic continuity, both methods have yet to produce chromosome level assemblies, as current research utilizing linkage mapping and telomeric assessment indicate that C. fimbriata has seven chromosomes (Fourie et al. 2019).

While there are similarities amongst the C. fimbriata genome assemblies, there are more noticeable differences between the genome annotations. The annotation of CMW14799 reflects these differences most prominently as 7,728 genes were predicted in early versions of the assembly and annotation before nanopore sequencing was incorporated (Fourie et al. 2019), when the number of predicted genes dropped to 7,274 (Fourie et al. 2020). The annotation of LPF1912, which followed an analogous annotation pipeline to the present study by using the Illumina based annotation of isolate CMW14799 and annotations from four other Ceratocystis species to train Augustus for gene prediction, had the greatest number of predicted genes with 8,042 gene models (Santos et al. 2020). These gene estimates seem most comparable to the 7,840 genes found within the Maker Max gene set of AS236; however, after filtering to remove low-quality predictions lacking evidence of transcript, protein family domain, and signal peptide domain support, only 6,481 gene models were kept in the final annotation. Although this estimate is more conservative than the former annotations, even the best gene prediction programs can still produce false or inaccurate predictions at the exon level (Yandell and Ence 2012). Thus, filtering the gene models to remove low-quality predictions, as has been seen in annotations of other fungi and plants (Campbell et al. 2014b, Pulman et al. 2016, Bowman et al. 2017) was considered a necessary step. This process also greatly reduced the number of hypothetical genes produced in the annotation as there are currently 236 hypothetical genes with unknown function, a third of which came from the genes reincorporated through the Signal-P pipeline.

Within plant pathology, there is a growing trend to use next generation or high-throughput sequencing technology to develop genomics-based diagnostic assays, as has been done repeatedly for virus, bacteria, and oomycete plant pathogens (Lang et al. 2010, De Beor and Lopez 2012, Withers et al. 2016, Maree et al. 2018, Rahman et al. 2019, Standish et al. 2022). With the C. fimbriata genome assembly produced in this study, the development of a sequence-based, culture independent diagnostic assay was possible. The use of comparative genomics of DNA-sequencing from C. manginecans and C. platani quickly and efficiently identified 148 species-specific markers within the C. fimbriata genome. This differs from the generation of diagnostic tools for other Ceratocystis spp., which involve assays developed around a limited number of polymorphic sites such as the rDNA ITS region or the ceratoplatanin gene (Yang and Juzwik 2017, Luchi et al. 2013, Heller and Keith 2018). The methods used in this study not only provide multiple genetic markers that could be used for other genomic comparisons but may also prove beneficial for limiting off-target amplification as demonstrated by recently developed C. fimbriata specific PCR primers designed within the rDNA ITS region (Kumari et al. 2021), that while sensitive to C. fimbriata were also able to amplify C. manginecans, C. acacivora, C. eucalypticola and C. fimbriatomima (Kumari et al. 2021).

Still, utilizing the species-specific markers identified in this study to develop a PCR-based assay is considered favorable for detection of C. fimbriata as it is slow to grow in culture (Stahr and Quesada-Ocampo 2019) and can be difficult and time consuming to isolate from plant hosts (Brito et al. 2019). Additionally, such assays can be more readily adopted by Plant Disease Clinics and research laboratories over serological methods that often require commercialization for wide-spread adoption to occur (De Beor and Lopez 2012). After PCR validation against diverse panels of C. fimbriata, Ceratocystis spp., other sweetpotato pathogens, and plant hosts, two genetic markers that solely amplified the C. fimbriata isolates remained. These markers are considered to be highly species specific and have the capability of detecting C. fimbriata isolated from a variety of plant hosts around the globe.

Being able to quickly and accurately diagnose a plant pathogen is an important first step to control plant diseases and help mitigate associated losses (De Boer and Lopez 2012). Sweetpotato black rot is considered such a devastating disease because of its ability to begin new infections and cause losses at multiple stages of production (Clark et al. 2009). The implementation of the C. fimbriata-specific molecular markers as a diagnostic tool would be of great benefit to sweetpotato growers and producers. The highly multi-copy marker T5G26 would be well suited for pathogen detection, even at low levels, within infected plant materials and soil samples (Kandal et al. 2015, Rojas et al. 2017). Tissue samples taken from sweetpotato slips could be tested for C. fimbriata, as a means to prevent transmission through contaminated seed (Harter 1913). Soil samples from sweetpotato fields could be tested for C. fimbriata presence prior to planting, enabling improved site selection and informed early fungicide applications to help protect young and developing sweetpotato roots when they are the most susceptible to infection (Parada-Rojas et al. 2021). Similarly, water from the dump tanks used to wash sweetpotatoes prior to packaging could be tested to alert growers as to when the water would need to be filtered and replaced, or if labeled fungicides should be applied later on in the packaging process. Also contemplated is a qPCR assay that would be even less time consuming than conventional PCR and could enable the use of spore quantification in experimental samples (Rahman et al. 2021, Standish et al. 2022). The species-specific diagnostic markers identified in this study represent a significant advancement towards improving sweetpotato black rot management.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Sequences

>maker-Cfim_AS236_tig05-augustus-gene-25.86-
mRNA-1 (Madison code: T5G26)
SEQ ID NO: 1
GCATGGCCGAATTTCTCATGAGCTTGTTGGTAGGTCATGTGTGCCTC
TATCGTTCTTTTGCCTCTGATGTATGGTAATGTATAGAGGTCGCCGG
TTTCGTTGAAGTAGGTCCGACGTGAACCTTTGCTTACGTATTGCACC
TGTTTCCTCTGCCACGACTTTGATTCCTTGTGCACAAATTCTTTTCA
CAGACAGTAAATTTTGATTAAGTCCTTCGACAAAGTACACTTCTATG
GTTACCGCATGTCCCCCCACATTAATCTCGGCATGTCCCATCGTTCT
GGTTTCCAATCGGAGTCCGCCCCAGTAGACCGGGATCGGATCTATTT
TTCGGGGACTGTGAATCTTCGTTAGATCGCCTGATAGATTGTCTGTG
CTGCCACTGTCTATTATCCAGTAGCTTGGGGTTTTCGTACCTGACGG
TGTTTTGTCCAAGTGTGTCGTGTTGGAGTGAATCGTCTTTTCAGACT
GCTTCTTCTTCGTTCTGCACTCATCTTCTTCGTGGCCAAGTTTGTGA
CACCAATCGCATTTCGGCCT
>snap_masked-Cfim_AS236_tig03-processed-gene-
10.27-mRNA-1 (Madison code: T3G9))
SEQ ID NO: 2
GCAAAGGGCAGCCGAATCACCTGGCTCTGATGGAGGAGGCAGAGAGG
AAAGGGGTGGATGTCGTATGCATTCAAGAACCGTATATAGGCAAACC
CTTAGAAGAAATGAGGACAGTCACACACCCGGCATACAAACTCATGA
CTCCACTAACGCAGTGGGACACGAGGCCGAGGGTGATGACATATGTC
AGAAACACAATAATGGCGACACTCGTACCCGTCTGTGACGGCGAACA
CGCAGATGCAGTCATAGTGGAACTGACAGGACAAGGCCTACACATCG
CCAATGTGTACAACGCCAACCCGCAAGACACCTCCCGAGGTATAGCA
TTGGCGCGGGCCCAGAAGACAGTCAAGGACAGAAAGATGATACTGTG
CGGTGACCTCAACCTACACTCACACATGTGGGACAGCAGGAGGAACG
AATCGCGAGATTGCTCATCGGTCCTGGACTGGCTAAGCGAGGACAAC
GTCACCCTGCTCAATGAGCCAGACGACAGCACATGTTTCCACAGCAA
GGAGTCCCCATCGGTGATCGACTTGGCCTTTGCCTCGAGCAACCTGC
TGGGCCAAAGCAGCTGCACGACCAAGGTCATGAAGGAGCTCAACTGT
GGCAGCGACCATTTCCCCCTCAGCACCACCATCGAAGGCATACAGAT
CGACGATGCAGTCAGAGCGACCCGGCACAACATGAACAGGCTCGACA
CAGAGAAGTTCACGAGGGCCTGCGCAAGAGAATCTACCTCTCTAAAC
GCAAGGGAACCCCTGAGGGCAGAGGACGTAGAAGAGCTCGCAGCCGG
GCTGGTCAAAGCGATGACCAAAGCACTCGACGAAGCGGCACCCAAGG
CCCTGGGCAGAGGCACAGGCAAGAGGTGGTGGAACCCGCAATGCACG
GGGGCGGTCAGAGAGATGAGGTCAGCATGGCGACAAACTGAAGCGGA
CAACTTCCCTGACGCGAGCTACGATGCGTACAAGGAGAAGAGAAGAG
CGTTCCAAGCTGAGATCTGAACGGCGAAACGGGAGCTATGGAGACAG
ACCATTGAGAACATGACGGATGCCACCGACGCGTTCAGGATGGTCAA
CCGACTGGGGAAAACAAACCCGGCCGGAGGACTCCCCCCGTTGACCC
AGGGCGGCACTACGAGAACCACACCGCAGGAGAAGGCCGAAGCCCTT
CTGGATACCCACACCCAGCCGGCGGAAGATTG

### Tables

TABLE 1
Isolate descriptions of Ceratocystis spp. DNA samples used for sequencing and/or lab validation
of the Ceratocystis fimbriata molecular diagnostic markers by polymerase chain reactions.

| | | | | | Year |
|---|---|---|---|---|---|
| Species | Isolate | Source | Host | Location | Collected |
| C. fimbriata* | AS236 | Quesada | Ipomoea batatas | North Carolina | 2015 |
| C. fimbriata* | C1421 | Harrington | Ipomoea batatas | North Carolina | 1998 |
| C. manginecans* | CMW 17570 | Marincowitz | Prosopsis | Oman, Africa | 2005 |
| C. platani* | CMW 48038 | Marincowitz | Platanus × acerifolia | Italy | 1995 |
| C. fimbriata | AS186 | Quesada | Ipomoea batatas | North Carolina | 2014 |
| C. fimbriata | MS567 | Nelson | Ipomoea batatas | Hawaii | — |
| C. fimbriata | MS571 | Quesada | Ipomoea batatas | North Carolina | 2018 |
| C. fimbriata | C4086 | Harrington | Ipomoea batatas | North Carolina | 2015 |
| C. fimbriata | C1418 | Harrington | Ipomoea batatas | North Carolina | 1998 |
| C. fimbriata | NRRL 13496 | ARS Collection | Coffea arabica | Suriname | — |
| C. fimbriata | CMW 42704 | Marincowitz | Ipomoea batatas | East Sabah, | 2014 |
| | | | | Malaysia | |
| C. fimbriata | CMW 42705 | Marincowitz | Ipomoea batatas | Southeast, | 2014 |
| | | | | Malaysia | |
| C. fimbriata | MS570 | Quesada | Ipomoea batatas | North Carolina | 2018 |
| C. fimbriata | MS572 | Quesada | Ipomoea batatas | North Carolina | 2018 |
| C. fimbriata | C1420 | Harrington | Ipomoea batatas | North Carolina | 1998 |
| C. fimbriata | C1421 | Harrington | Ipomoea batatas | North Carolina | 1998 |
| C. fimbriata | C1422 | Harrington | Ipomoea batatas | North Carolina | 1998 |
| C. fimbriata | LPF 1336 | Alfenas | Ficus carica | São Paulo | — |
| C. fimbriata | LPF1394 | Alfenas | Hevea brasiliensis | Acre | — |
| C. fimbriata | LPF 1323 | Alfenas | Gmelina arborea | Pará | — |
| C. fimbriata | LPF 1489 | Alfenas | Caryocar brasiliense | Minas Gerais | — |
| C. fimbriata | LPF 1156 | Alfenas | Mangifera indica | Ceará | — |
| | | | ‘Espada’ | | |
| C. fimbriata | LPF1443 | Alfenas | Actinidia deliciosa | Rio Grande do | — |
| | | | | Sul | |
| C. fimbriata | LPF 1331 | Alfenas | Annona squamosa | Rio de Janeiro | — |
| C. fimbriata | SBS-1 | Alfenas | Eucalyptus spp. | Bahia | — |
| C. fimbriata | LPF 1560 | Alfenas | Carapa guianensis | Acre | — |
| C. fimbriata | LPF2199 | Alfenas | Tectona grandis | Mato Grosso | — |
| C. fimbriata | AS527 | Quesada | Ipomoea batatas | Canada | 2016 |
| C. betulina | C1709 | Trouillas, | Betula platphylla | Japan | 2000 |
| | | Harrington | | | |
| C. tiliae | C1954 | Trouillas, | Tilia americana | Iowa | 2002 |
| | | Harrington | | | |
| C. harringtonii | C685 | Trouillas, | Populus tremuloides | Quebec, Canada | 1993 |
| | | Harrington | | | |
| C. variospora | C1009 | Trouillas, | Quercus ellipsoidalis | Minnesota | 1995 |
| | | Harrington | | | |
| C. caryae | C1827 | Trouillas, | Carya ovata | Iowa | 2001 |
| | | Harrington | | | |
| C. smalleyi | C684 | Trouillas, | Carya cordiformis | Wisconsin | 1986 |
| | | Harrington | | | |
| C. destructans | KARE1448 | Trouillas | Prunus dulcis | California | 2016 |
| C. cacaofunesta | LPF 1332 | Alfenas | Theobroma cacao | Bahia | — |
| C. paradoxa | MS617 | Raid | Saccharum officinarum | Florida | 2020 |

*Denotes the Ceratocystis fimbriata, C. manginecans, and C. platani isolates that were used to generate all sequence files in addition to their use for lab validation of the C. fimbriata molecular diagnostic markers by polymerase chain reactions.

TABLE 2
Isolate descriptions of sweetpotato pathogen DNA samples used for lab validation of the
Ceratocystis fimbriata molecular diagnostic markers by polymerase chain reactions.

| | Organism | | | | Location | Year |
|---|---|---|---|---|---|---|
| Species | Type | Isolate Code | Source | Host | Origin | Collected |
| Bretziella | fungus | — | Burkhardt | Quercus | California | — |
| fagacearum | | | | rubra | | |
| Ophiastoma | fungus | NRRL 6404 | ARS | — | — | — |
| ulmi | | | Collection | | | |
| Geotrichum | fungus | MS614 | Quesada | Ipomoca | North | 2021 |
| candidatum | | | | batatas | Carolina | |
| Geotrichum | fungus | MS615 | Quesada | Ipomoea | North | 2021 |
| candidatum | | | | batatas | Carolina | |
| Rhizopus | fungus | AS046 | Quesada | Ipomoea | North | 2015 |
| stolonifer | | | | batatas | Carolina | |
| Rhizopus | fungus | SW003 | Quesada | Ipomoea | North | 2013 |
| stolonifer | | | | batatas | Carolina | |
| Sclerotium | fungus | MS568 | Quesada | Ipomoea | North | 2018 |
| rolfsii | | | | batatas | Carolina | |
| Sclerotium | fungus | MS569 | Quesada | Ipomoea | North | 2018 |
| rolfsii | | | | batatas | Carolina | |
| Fusarium | fungus | AS088 | Quesada | Ipomoea | North | 2014 |
| acuminatum | | | | batatas | Carolina | |
| Fusarium | fungus | AS217 | Quesada | Ipomoea | North | 2015 |
| solani | | | | batatas | Carolina | |
| Fusarium | fungus | G2 | Clark | Ipomoea | Louisianna | 2011 |
| oxysporum | | | | batatas | | |
| Fusarium | fungus | G7 | Clark | Ipomoea | Louisianna | 2011 |
| proliferatum | | | | batatas | | |
| Fusarium | fungus | G89-01 | Clark | Ipomoea | Louisianna | 1989 |
| denticulatum | | | | batatas | | |
| Dickeya | bacteria | MS594 | Quesada | Ipomoea | North | 2019 |
| dadantii | | | | batatas | Carolina | |
| Streptomyces | bacteria | 90-1 | Williamson | Ipomoea | Texas | — |
| ipomoeae | | | | batatas | | |
| Streptomyces | bacteria | L81-44 | Williamson | Ipomoea | Louisianna | — |
| ipomoeae | | | | batatas | | |
| Meloidogyne | nematode | 18-6908 | Gorny | Ipomoea | North | 2017 |
| enterolobii | | | | batatas | Carolina | |

TABLE 3
Plant species and varieties used for lab validation
of the Ceratocystis fimbriata molecular diagnostic
markers by polymerase chain reaction.

| Species | Crop Type | Cultivar |
|---|---|---|
| Ipomoea batatas | Sweetpotato | Covington |
| Ipomoea nil | Morning Glory | Scarlett O’Hara |
| Daucus carota | Carrot | Danvers 126 |
| Nicotiana tabacaum | Tobacco | K326 |
| Glycine max | Soybean | AG48X7 |
| Gossypium hirsutum | Cotton | Deltapine 1538 |
| Lactuca sativa | Lettuce | Sparx |
| Triticum aestivum | Wheat | Dynagrow Shirley |
| Brassica oleracea | Cabbage | Ruby Ball |
| Cucurbita maxima | Squash | Multipik |
| Citrullus lanatus | Watermelon | Crimson Sweet |
| Arachis hypogaea | Peanut | Bailey |

TABLE 4
Genome assembly statistics for Ceratocystis fimbriata. BUSCO
coverage estimates are based on comparison to the fungi specific lineage
database (fungi_odb). The full number of predicted gene models
during genome annotation through Maker is represented as the
Maker Max gene set, while the Maker Standard gene set represents
only gene predictions with evidence of transcript, protein family
domain, or signal peptide domain support.

| Assembly size | 31,692,598 bp |
|---|---|
| Percent GC Content | 48.27% |
| Total Number of Contigs | 18 |
| Largest Contig | 9,655,333 |
| Contig N50 | 3,836,501 |
| Coverage of complete fungal BUSCO genes | 98.4% |
| Predicted Genes: Maker Max/Maker Standard | 7,840/6,481 |

TABLE 5
Candidate Gene IDs, exon number from gff file,
gene copy number, functional annotation, PCR
primer sequences, and expected product sizes
after polymerase chain reaction for the two
final Ceratocystis fimbriata species-specific
diagnostic candidate markers. Gene copy
numbers were determined through a self-BLASTP
alignment of the C. fimbriata protein sequences.

| | | | | | Pro- |
|---|---|---|---|---|---|
| Can- | | | | Primer | duct |
| didate | | Gene | Functional | Sequence | Size |
| Gene | Exon | Copy | Annotation | (5′ to 3′) | (bp) |
| T3G9 | 684 | 21 | Endonuclease- | F-GAGGAAAG | 1042 |
| | | | reverse | GGGTGGATGT | |
| | | | transcriptase | CG (SEQ ID | |
| | | | | NO: 3) | |
| | | | | R-TCGGTTGA | |
| | | | | CCATCCTGAA | |
| | | | | CG (SEQ ID | |
| | | | | NO: 4) | |
| T5G26 | 1347 | 45 | Expressed | F-TCGCCGGT | 433 |
| | | | gene of | TTCGTTGAAG | |
| | | | unknown | TA (SEQ ID | |
| | | | function | NO: 5) | |
| | | | | R-GTGTCACA | |
| | | | | AACTTGGCCA | |
| | | | | CG (SEQ ID | |
| | | | | NO: 6) | |

### REFERENCES

Anders, S., Pyl, P. T., and Huber, W. 2015. HTSeq—a Python framework to work with high-throughput sequencing data. Bioinformatics. 31:166-169.
Baker, C. J., Harrington, T. C., Krauss, U., and Alfenas, A. C. 2003. Genetic Variability and Host Specialization in the Latin American Clade of Ceratocystis fimbriata. Phytopathology. 93:1274-1284.
Barnes, I., Fourie, A., Wingfield, M. J., Harrington, T. C., McNew, D. L., Sugiyama, L. S., Luiz, B. C., Heller, W. P., Keith L. M., 2018. New Ceratocystis species associated with rapid death of Metrosideros polymorpha in Hawai'i. Persoonia—Molecular Phylogeny and Evolution of Fungi. 40:154-181.
de Beer, Z. W., Duong, T. A., Barnes, I., Wingfield, B. D., and Wingfield, M. J. 2014. Redefining Ceratocystis and allied genera. Studies in Mycology. 79:187-219.
Bickers, C. 2015. Sweet potatoes: Juice, pet food products could increase market demand. Southeast Farm Press. p. 4.
Bolger, A. M., Lohse, M., and Usadel, B. 2014. Trimmomatic: a flexible trimmer for Illumina sequence data. Bioinformatics. 30:2114-2120.
Bowman, M. J., Pulman, J. A., Liu, T. L., and Childs, K. L. 2017. A modified GC-specific MAKER gene annotation method reveals improved and novel gene predictions of high and low GC content in Oryza sativa. BMC Bioinformatics. 18:522
Brito, R. A. S., Cavalcante, G. P., Borel, F. C., and Maffia, L. A. 2019. Detection and isolation of Ceratocystis fimbriata in mango trees on semi-selective medium. European Journal of Plant Pathology. 155:667-669.
CAB International. 2020. Ceratocystis fimbriata (Ceratocystis blight). Crop Protection Compendium.
Campbell, M. S., Holt, C., Moore, B., and Yandell, M. 2014a. Genome Annotation and Curation Using MAKER and MAKER-P. Current Protocols in Bioinformatics. 48: 4.11.1-4.11.39
Campbell, M. S., Law, M., Holt, C., Stein, J. C., Moghe, G. D., Hufnagel, D. E., et al. 2014b. MAKER-P: A Tool Kit for the Rapid Creation, Management, and Quality Control of Plant Genome Annotations. Plant Physiology. 164:513-524.
Cerqueira, G. C., Arnaud, M. B., Inglis, D. O., Skrzvpek, M. S., Binkley, G., Simison, M., et al. 2014. The Aspergillus Genome Database: multispecies curation and incorporation of RNA-Seq data to improve structural gene annotations. Nucleic Acids Res. 42:D705-D710.
Cherry, J. M., Hong, E. L., Amundsen, C., Balakrishnan, R., Binkley, G., Chan, E. T., Christie, K. R., Costanzo, M. C., Dwight, S. S., Engel, S. R., Fisk, D. G., Hirschman, J. E., Hitz, B. C., Karra, K., Krieger, C. J., Miyasato, S. R., Nash, R, S., Park, J., Skrzypek, M. S., Simison, M., Weng, S., Wong, E. D., 2012. Saccharomyces Genome Database: the genomics resource of budding yeast. Nucleic Acids Res. 40:D700-D705.
Clark, C. A., Holmes, G. J., and Ferrin, D. M. 2009. Major Fungal and Bacterial Diseases. In The Sweetpotato, eds. G. Loebenstein and G. Thottappilly., p. 81-104.
Dobin, A., and Gingeras, T. R. 2015. Mapping RNA-seq Reads with STAR. Curr Protoc Bioinformatics. 51:11.14.1-11.14.19.
Eddy, S. R. 2018. HMMER: biosequence analysis using profile hidden Markov models. HMMER.
El Sheikha, A. F., and Ray, R. C. 2017. Potential impacts of bioprocessing of sweet potato: Review. Critical Reviews in Food Science and Nutrition. 57:455-471.
Elbers, J. P., Rogers, M. F., Perelman, P. L., Proskuryakova, A. A., Serdyukova, N. A., Johnson, W. E., et al. 2019. Improving Illumina assemblies with Hi-C and long reads: An example with the North African dromedary. Molecular Ecology Resources. 19:1015-1026.
Engelbrecht, C. J. B., and Harrington, T. C. 2005. Intersterility, morphology and taxonomy of Ceratocystis fimbriata on sweet potato, cacao and sycamore. Mycologia. 97:57-69.
Food and Agriculture Organization of the United Nations. 2019. FAOSTAT Data, Sweet potatoes.
Fourie, A., de Jonge, R., van der Nest, M. A., Duong, T. A., Wingfield, M. J., Wingfield, B. D., et al. 2020. Genome comparisons suggest an association between Ceratocystis host adaptations and effector clusters in unique transposable element families. Fungal Genetics and Biology. 143:103433.
Fourie, A., van der Nest, M. A., de Vos, L., Wingfield, M. J., Wingfield, B. D., and Barnes, I. 2019. QTL mapping of mycelial growth and aggressiveness to distinct hosts in Ceratocystis pathogens. Fungal Genetics and Biology. 131:103242.
Galagan, J. E., Calvo, S. E., Borkovich, K. A., Selker, E. U., Read, N. D., Jaffe, D., FitzHugh, W., Ma, L., Smimov, S., Purcell, S., Rehman, B., Elkins, T., Engels, R., Wang, S., Nielsen, C. B., Butler, J., Endrizzi, M., Qui, D., Ianakiev, P., Bell-Pedersen, D., Nelson, M. A., Wemer-Washbume, M., Selitrennikoff, C. P., Kinsey, J. A., Braun, E. L., Zelter, A., Schulte, U., Kothe, G. O., Jedd, G., Mewes, W., Staben, C., Marcotte, E., Greenberg, D., Roy, A., Foley, K., Naylor, J., Stange-Thomann, N., Barrett, R., Gnerre, S., Kamal, M., Kamvysselis, M., Mauceli, E., Bielke, C., Rudd, S., Frishman, D., Krystofova, S., Rasmussen, C., Metzenberge, R. L.,
Perkins, D. D., Kroken, S., Cogoni, C., Macino, G., Catcheside, D., Li, W., Pratt, R. J., Osmani, S. A., DeSouza, C. P. C., Glass, L., Orbach, M. J., Berglund, J. A., Voelker, R., Yarden, O., Plamann, M., Seiler, S., Dunlap, J., Radford, A., Aramayo, R., Natvig, D. O., Alex, L. A., Mannhaupt, G., Ebbole, D. J., Freitag, M., Paulsen, I., Sachs, M. S., Lander, E. S., Nusbaum, C., Birren, B., 2003. The genome sequence of the filamentous fungus Neurospora crassa. Nature. 422:859-868.
Grabherr, M. G., Haas, B. J., Yassour, M., Levin, J. Z., Thompson, D. A., Amit, I., et al. 2011. Trinity: reconstructing a full-length transcriptome without a genome from RNA-Seq data. Nat Biotechnol. 29:644-652.
Harrington, T. C., Thorpe, D. J., and Alfenas, A. C. 2011. Genetic Variation and Variation in Aggressiveness to Native and Exotic Hosts Among Brazilian Populations of Ceratocystis fimbriata. Phytopathology. 101:555-566.
Harter, L. L. 1913. Control of the Black-rot and Stem-rot of the sweet potato. US Dept. of Ag Bureau of Plant Industry. Circ No 114:15-18.
Harter, L. L. 1916. Sweet-Potato Diseases. US Dept of Ag Farmers' Bull. 714:8-11.
Heller, W. P., and Keith, L. M. 2018. Real-Time PCR Assays to Detect and Distinguish the Rapid ‘Ohi’a Death Pathogens Ceratocystis lukuohia and C. huliohia. Phytopathology. 108:1395-1401.
Ho, E. C., Cahill, M. J., and Saville, B. J. 2007. Gene discovery and transcript analyses in the corn smut pathogen Ustilago maydis: expressed sequence tag and genome sequence comparison. BMC Genomics. 8:334.
Holland, L. A., Lawrence, D. P., Nouri, M. T., Travadon, R., Harrington, T. C., and Trouillas, F. P. 2019. Taxonomic Revision and Multi-locus Phylogeny of the North American Clade Of Ceratocystis. Fungal Systematics and Evolution. 3:135-156.
Ison, J. C., Rice, P. M., and Bleasby, A. J. 2011. EMBOSS Developer's Guide: Bioinformatics Programming. Cambridge, United Kingdom: Cambridge University Press.
Kandel, Y. R., Haudenshield, J. S., Srour, A. Y., Islam, K. T., Fakhoury, A. M., Santos, P., et al. 2015. Multilaboratory Comparison of Quantitative PCR Assays for Detection and Quantification of Fusarium virguliforme from Soybean Roots and Soil. Phytopathology. 105:1601-1611.
Kaur, S., Anh, Q., and Lynn, and. High quality DNA from Fusarium oxysporum conidia suitable for library preparation and long read sequencing with PacBio v4.
Keith, L. M., Hughes, R. F., Sugiyama, L. S., Heller, W. P., Bushe, B. C., and Friday, J. B. 2015. First Report of Ceratocystis Wilt on ‘Ohi’a (Metrosideros polymorpha). Plant Disease. 99:1276.
Koren, S., Walenz, B. P., Berlin, K., Miller, J. R., Bergman, N. H., and Phillippy, A. M. 2017. Canu: scalable and accurate long-read assembly via adaptive k-mer weighting and repeat separation. Genome Research. 27:722-736.
Koressaar, T., and Remm, M. 2007. Enhancements and modifications of primer design program Primer3. Bioinformatics. 23:1289-1291.
Korf, I. 2004. Gene finding in novel genomes. BMC Bioinformatics: 9.
Kumari, N., Shukla, P. K., Singh, H., Fatima, T., Bajpai, A. 2021. Development of species-specific PCR based detection assay for Ceratocvstis fimbriata, mango wilt pathogen. Indian Phytopathology. https://doi.org/10.1007/s42360-021-00438-9
Lang, J. M., Hamilton, J. P., Diaz, M. G. Q., Van Sluys, M. A., Burgos, Ma. R. G., Vera Cruz, C. M., et al. 2010. Genomics-Based Diagnostic Marker Development for Xanthomonas oryzae pv. oryzae and X. oryzae pv. oryzicola. Plant Disease. 94:311-319.
Langmead, B., Wilks, C., Antonescu, V., and Charles, R. 2019. Scaling read aligners to hundreds of threads on general-purpose processors ed. John Hancock. Bioinformatics. 35:421-432.
Luchi, N., Ghelardini, L., Belbahri, L., Quartier, M., and Santini, A. 2013. Rapid Detection of Ceratocystis platani Inoculum by Quantitative Real-Time PCR Assay. Appl Environ Microbiol. 79:5394-5404.
Maree, H. J., Fox, A., Al Rwahnih, M., Boonham, N., and Candresse, T. 2018. Application of HTS for Routine Plant Virus Diagnostics: State of the Art and Challenges. Front Plant Sci. 9
Parada-Rojas, C. H., Pecota, K., Almeyda, C., Yencho, G. C., and Quesada-Ocampo, L. M. 2021. Sweetpotato root development influences susceptibility to black rot caused by the fungal pathogen Ceratocystis fimbriata. Phytopathology. FIRST LOOK.
Passera, A., Marcolungo, L., Casati, P., Brasca, M., Quaglino, F., Cantaloni, C., et al. 2018. Hybrid genome assembly and annotation of Paenibacillus pasadenensis strain R16 reveals insights on endophytic life style and antifungal activity ed. Lorenzo Brusetti. PLOS ONE. 13:e0189993.
Petersen, T. N., Brunak, S., von Heijne, G., and Nielsen, H. 2011. SignalP 4.0: discriminating signal peptides from transmembrane regions. Nature Methods. 8:785-786.
Polonio, Á., Diaz-Martinez, L., Fernández-Ortuño, D., de Vicente, A., Romero. D., López-Ruiz, F. J., et al. 2021. A Hybrid Genome Assembly Resource for Podosphaera xanthii, the Main Causal Agent of Powdery Mildew Disease in Cucurbits. Molecular Plant-Microbe Interactions. 34:319-324.
Pulman, J. A., Childs, K. L., Sgambelluri, R. M., and Walton, J. D. 2016. Expansion and diversification of the MSDIN family of cyclic peptide genes in the poisonous agarics Amanita phalloides and A. bisporigera. BMC Genomics. 17:1038
Rahman, A., Góngora-Castillo, E., Bowman, M. J., Childs, K. L., Gent, D. H., Martin, F. N., Quesada-Ocampo, L. M., 2019. Genome Sequencing and Transcriptome Analysis of the Hop Downy Mildew Pathogen Pseudoperonospora hunuli Reveal Species-Specific Genes for Molecular Detection. Phytopathology. 109:1354-1366.
Rahman, A., Standish, J. R., D'Arcangelo, K. N., and Quesada-Ocampo, L. M. 2021. Clade-Specific Biosurveillance of Pseudoperonospora cubensis Using Spore Traps for Precision Disease Management of Cucurbit Downy Mildew. Phytopathology. 111:312-320.
Ray, R. C., and Ravi, V. 2005. Post Harvest Spoilage of Sweetpotato in Tropics and Control Measures. Critical Reviews in Food Science and Nutrition. 45:623-644.
Rojas, J. A., Miles, T. D., Coffey, M. D., Martin, F. N., and Chilvers, M. I. 2017. Development and Application of qPCR and RPA Genus- and Species-Specific Detection of Phytophthora sojae and P. sansomeana Root Rot Pathogens of Soybean. Plant Disease. 101:1171-1181.
Santini, A., and Capretti, P. 2000. Analysis of the Italian population of Ceratocystis fimbriata f.sp. platani using RAPD and minisatellite markers. Plant Pathology. 49:461-467.
Santos, S. A., Vidigal, P. M. P., Thrimawithana, A., Betancourth, B. M. L., Guimarães, L. M. S., Templeton, M. D., Alfenas, A. C., 2020. Comparative genomic and transcriptomic analyses reveal different pathogenicity-related genes among three eucalyptus fungal pathogens. Fungal Genetics and Biology. 137:103332.
Scott, G. J. 1992. Sweet potatoes as animal feed in developing countries: present patterns and future prospects. In Roots, tubers, plantains, and bananas in animal feeding: proceedings of the FAO Expert Consultation, FAO animal production and health paper, eds. David Machin and Solveig Nyvold. CIAT, Cali, Colombia: Food and Agriculture Organization of the United Nations.
Scruggs, A. C., Basaiah, T., Adams, M. L., and Quesada-Ocampo, L. M. 2017. Genetic Diversity, Fungicide Sensitivity, and Host Resistance to Ceratocystis fimbriata Infecting Sweetpotato in North Carolina. Plant Disease. 101:994-1001.
Simao, F. A., Waterhouse, R. M., Ioannidis, P., Kriventseva, E. V., and Zdobnov, E. M. 2015. BUSCO: assessing genome assembly and annotation completeness with single-copy orthologs. Bioinformatics. 31:3210-3212.
Species Fungorum—Species synonymy. Ceratocystis fimbriata Ellis & Halst.
Stahr, M. N., and Quesada-Ocampo, L. M. 2019. Black Rot of Sweetpotato: A Comprehensive Diagnostic Guide. Plant Health Progress. 20:255-260.
Stahr, M., and Quesada-Ocampo, L. M. 2020. Assessing the Role of Temperature, Inoculum Density, and Wounding on Disease Progression of the Fungal Pathogen Ceratocystis fimbriata Causing Black Rot in Sweetpotato. Plant Disease. 104:930-937.
Stahr, M. N., and Quesada-Ocampo, L. M. 2021. Effects of Water Temperature, Inoculum Concentration and Age, and Sanitizers on Infection of Ceratocystis fimbriata, Causal Agent of Black Rot in Sweetpotato. Plant Disease. PDIS-07-20-1475.
Standish, J. R., Góngora-Castillo, E., Bowman, M., Childs, K., Tian, M., and Quesada-Ocampo, L. M. 202X. Development, validation, and utility of species-specific diagnostic markers for detection of Peronospora belbahrii. Phytopathology. Accepted.
Stanke, M., and Waack, S. 2003. Gene prediction with a hidden Markov model and a new intron submodel. Bioinformatics. 19:ii215-ii225.
Trapnell, C., Williams, B. A., Pertea, G., Mortazavi, A., Kwan, G., van Baren, M. J., Salzberg, S. L., Wold, B. J., Pachter, L., 2010. Transcript assembly and quantification by RNA-Seq reveals unannotated transcripts and isoform switching during cell differentiation. Nature Biotechnology. 28:511-515.
Vaser, R., Sović, I., Nagarajan, N., and Ŝikić, M. 2017. Fast and accurate de novo genome assembly from long uncorrected reads. Genome Res. 27:737-746.
Walker, B. J., Abeel, T., Shea, T., Priest, M., Abouelliel, A., Sakthikumar, S., Cuomo, C. A., Seng, Q., Wortman, J., Young, S. K., Earl, A. M., Wang, J., 2014. Pilon: An Integrated Tool for Comprehensive Microbial Variant Detection and Genome Assembly Improvement ed. Junwen Wang. PLoS ONE. 9:e112963.
Waterhouse, R. M., Seppey, M., Simao, F. A., Manni, M., Ioannidis, P., Klioutchnikov, G., Kriventseva, E. V., Zdobnov, E. M., 2018. BUSCO Applications from Quality Assessments to Gene Prediction and Phylogenomics. Molecular Biology and Evolution. 35:543-548.
Webster, R. K., and Butler, E. E. 1967. A morphological and biological concept of the species Ceratocystis fimbriata. Canadian Journal of Botany. 45:1457-1468.
Wilken. P. M., Steenkamp, E. T., Wingfield, M. J., Wilhelm de Beer, Z., and Wingfield, B. D. 2013. Draft nuclear genome sequence for the plant pathogen, Ceratocystis fimbriata. IMA Fungus. 4:357-358.
Wingfield, B. D., Fourie, A., Simpson, M. C., Bushula-Njah, V. S., Aylward, J., Barnes, I., Coetzee, M. P. A., Dreyer, L. L., Duong, T. A., Geiser, D. M., Roets, F., Steenkamp, E. T., van der Nest, M. A., van Heerden, C. J., Wingfield, M. J., 2019. IMA Genome-F 11: Draft genome sequences of Fusarium xylariodes, Tetrasphearia gauchensis and T. zuluensis and genome annotation for Ceratocystis fimbriata. IMA Fungus. 10:13.
Withers, S., Gongora-Castillo, E., Gent, D., Thomas, A., Ojiambo, P. S., and Quesada-Ocampo, L. M. 2016. Using Next-Generation Sequencing to Develop Molecular Diagnostics for Pseudoperonospora cubensis, the Cucurbit Downy Mildew Pathogen. Phytopathology. 106:1105-1116.
Yandell, M., and Ence, D. 2012. A beginner's guide to eukaryotic genome annotation. Nature Reviews Genetics. 13:329-342.
Yang, A., and Juzwik, J. 2017. Use of Nested and Real-Time PCR for the Detection of Ceratocystis fagacearum in the Sapwood of Diseased Oak Species in Minnesota. Plant Disease. 101:480-486.
Yates, A. D., Achuthan, P., Akanni, W., Allen, J., Allen, J., Alvarez-Jarreta, J., Armean, I. M., Azov, A. G., Bennet, R., Bhai, J., Billis, K., Bodda, S., Marugan, J. C., Cummins, C., Davidson, C., Dodiya, K., Fatima, R., Gall, A., Giron, C. G., Gil, L., Grego, T., Haggerty, L., Haskell, E., Hourlier, T., Izuogu, O. G., Janacek, S. H., Juettemann, T., Kay, M., Lavidas, I., Le, T., Lemos, D., Martinez, J. G., Maurel, T., McDowall, M., McMahon, A., Mohanan, S., Moore, B., Nuhn, M., Oheh, D. N., Parker, A., Parton, A., Patricio, M., Sakthivel, M. P., Salem, A. I. A., Schmitt, B. M., Schuilenburg, H., Sheppard, D., Sycheva, M., Szuba, M., Taylor, K., Thomann, A., Threadgold, G., Vullo, A., Walts, B., Winterbottom, A., Zadissa, A., Chakiachvili, M., Flint, B., Frankish, A., Hunt, S. E., Ilsley, G., Kostadima, M., Langridge, N., Loveland, J. E., Martin, F. J., Morales, J., Mudge, J. M., Muffato, M., Perry, E., Ruffier, M., Trevanion, S. J., Cunningham, F., Howe, K. L., Zebrino, D. R., Flicek, P., 2019. Ensembl 2020. Nucleic Acids Research.

1. A method of treating sweetpotatoes infected with Ceratocystis fimbriata, the method comprising amplifying nucleic acid using primers which amplify a region of SEQ ID NO: 1 and/or SEQ ID NO: 2, and detecting the amplified nucleic acid, thereby detecting Ceratocystis fimbriata, and treating the sweetpotatoes to reduce or eliminate Ceratocystis fimbriata.

2. The method of claim 1, wherein said primers are 90% or more identical to SEQ ID NOS: 5 and 6.

3. The method of claim 1, wherein said primers are 90% or more identical to SEQ ID NOS: 3 and 4.

4. The method of claim 1, wherein after detection, sweetpotatoes are treated with an antifungal.

5. The method of claim 4, wherein the antifungal is specific for Ceratocystis fimbriata.

6. The method of claim 1, wherein the primers do not amplify nucleic acid from other Ceratocystis species.

7. The method of claim 6, wherein the primers do not amplify nucleic acid from Ceratocystis manginecans or Ceratocystis platari.

8. An isolated nucleic acid, wherein the isolated nucleic acid hybridizes with at least 5 consecutive nucleic acids, in a 5′ to 3′ or 3′ to 5′ direction, within SEQ ID NO: 1 or 2.

9. The isolated nucleic acid of claim 8, wherein the nucleic acid is at least 10 nucleotides in length, but not more than 50 nucleotides in length.

10. The isolated nucleic acid of claim 8, wherein the isolated nucleic acid is 90% or more complementary to SEQ ID NO: 1 or 2 along the isolated nucleic acid's length.

11. The isolated nucleic acid of claim 10, wherein the isolated nucleic acid is 90% or more identical to SEQ ID NO: 3.

12. The isolated nucleic acid of claim 10, wherein the isolated nucleic acid is 90% or more identical to SEQ ID NO: 4.

13. The isolated nucleic acid of claim 10, wherein the isolated nucleic acid is 90% or more identical to SEQ ID NO: 5.

14. The isolated nucleic acid of claim 10, wherein the isolated nucleic acid is 90% or more identical to SEQ ID NO: 6.

15. A kit comprising at least one oligomer capable of hybridizing in a 5′ to 3′ or 3′ to 5′ direction within SEQ ID NO: 1 or SEQ ID NO: 2.

16. The kit of 15, wherein the kit further comprises additional reagents for amplification or detection of nucleic acid.

17. The kit of claim 16, wherein the kit additional reagents can comprise one or more of labeling reagents, buffers, or enzymes.

18. The kit of claim 17, wherein enzymes can comprise one or more of DNA polymerase or reverse transcriptase.

19. (canceled)

20. (canceled)

21. (canceled)

22. (canceled)

23. (canceled)


SEQUENCE LISTING (6 sequences)

SEQ ID NO: 1 - DNA, 537 bp, Ceratocystis fimbriata
  source 1..537: mol_type = other DNA
gcatggccgaatttctcatgagcttgttggtaggtcatgtgtgcctctatcgttcttttgcctctgatgtatggtaatgtatagaggtcgccggtttcgttgaagtaggtccgacgtgaacctttgcttacgtattgcacctgtttcctctgccacgactttgattccttgtgcacaaattcttttcacagacagtaaattttgattaagtccttcgacaaagtacacttctatggttaccgcatgtccccccacattaatctcggcatgtcccatcgttctggtttccaatcggagtccgccccagtagaccgggatcggatctatttttcggggactgtgaatcttcgttagatcgcctgatagattgtctgtgctgccactgtctattatccagtagcttggggttttcgtacctgacggtgttttgtccaagtgtgtcgtgttggagtgaatcgtcttttcagactgcttcttcttcgttctgcactcatcttcttcgtggccaagtttgtgacaccaatcgcatttcggcct

SEQ ID NO: 2 - DNA, 1207 bp, Ceratocystis fimbriata
  source 1..1207: mol_type = other DNA
gcaaagggcagccgaatcacctggctctgatggaggaggcagagaggaaaggggtggatgtcgtatgcattcaagaaccgtatataggcaaacccttagaagaaatgaggacagtcacacacccggcatacaaactcatgactccactaacgcagtgggacacgaggccgagggtgatgacatatgtcagaaacacaataatggcgacactcgtacccgtctgtgacggcgaacacgcagatgcagtcatagtggaactgacaggacaaggcctacacatcgccaatgtgtacaacgccaacccgcaagacacctcccgaggtatagcattggcgcgggcccagaagacagtcaaggacagaaagatgatactgtgcggtgacctcaacctacactcacacatgtgggacagcaggaggaacgaatcgcgagattgctcatcggtcctggactggctaagcgaggacaacgtcaccctgctcaatgagccagacgacagcacatgtttccacagcaaggagtccccatcggtgatcgacttggcctttgcctcgagcaacctgctgggccaaagcagctgcacgaccaaggtcatgaaggagctcaactgtggcagcgaccatttccccctcagcaccaccatcgaaggcatacagatcgacgatgcagtcagagcgacccggcacaacatgaacaggctcgacacagagaagttcacgagggcctgcgcaagagaatctacctctctaaacgcaagggaacccctgagggcagaggacgtagaagagctcgcagccgggctggtcaaagcgatgaccaaagcactcgacgaagcggcacccaaggccctgggcagaggcacaggcaagaggtggtggaacccgcaatgcacgggggcggtcagagagatgaggtcagcatggcgacaaactgaagcggacaacttccctgacgcgagctacgatgcgtacaaggagaagagaagagcgttccaagctgagatctgaacggcgaaacgggagctatggagacagaccattgagaacatgacggatgccaccgacgcgttcaggatggtcaaccgactggggaaaacaaacccggccggaggactccccccgttgacccagggcggcactacgagaaccacaccgcaggagaaggccgaagcccttctggatacccacacccagccggcggaagattg

SEQ ID NO: 3 - DNA, 20 bp, Ceratocystis fimbriata
  source 1..20: mol_type = other DNA
gaggaaaggggtggatgtcg

SEQ ID NO: 4 - DNA, 20 bp, Ceratocystis fimbriata
  source 1..20: mol_type = other DNA
tcggttgaccatcctgaacg

SEQ ID NO: 5 - DNA, 20 bp, Ceratocystis fimbriata
  source 1..20: mol_type = other DNA
tcgccggtttcgttgaagta

SEQ ID NO: 6 - DNA, 20 bp, Ceratocystis fimbriata
  source 1..20: mol_type = other DNA
gtgtcacaaacttggccacg